(19) United States
(12) Patent Application Publication
(10) Pub. No.: US 2021/0133896 A1
(43) Pub. Date: May 6, 2021

(54) MOBILE FITNESS SOCIAL MEDIA COMPETITION PLATFORM

(71) Applicants: Matthew Vincent Magariello, Robbinsville, NJ (US); Jessica Wortelmann, Robbinsville, NJ (US)

(72) Inventors: Matthew Vincent Magariello, Robbinsville, NJ (US); Jessica Wortelmann, Robbinsville, NJ (US)

(21) Appl. No.: 16/668,541

(22) Filed: Oct. 30, 2019

(51) Int. Cl. G06Q 50/00; G16H 20/30; G07F 17/32

(52) U.S. Cl. CPC G06Q 50/01; G16H 20/30; G06Q 20/065; G07F 17/3251; H04L 2209/56; G07F 17/3288

(57) ABSTRACT

A method of operating an online fitness-related social media platform over a network includes the following: providing a server of a social media platform system; providing, by the server, an application for execution on a user device connected to the network and associated with a user, the user device being remote from the server; providing a tracking functionality adapted to track fitness data for each user device, providing a ranking functionality adapted to rank one or more values associated with the fitness data; and operating the social media platform system in a competition between users wherein their fitness data is tracked and ranked accordingly.

### BACKGROUND OF THE INVENTION

The present invention relates to online fitness-based platforms and, more particularly, to a mobile fitness social media competition platform.

No one knows who the strongest people, pound for pound, on the planet are at any given time. No one knows the strongest athletes in any sport at any given time. Supplement companies are plentiful, but with no one platform on which to advertise their products. The problems with current software applications in the fitness field is that none of them track and rank athletes across the planet in a competitive manner, and so miss an ideal platform for being a practical application for improving the physical health of its user, and by extension being an online space for promoting supplements and other fitness products. Furthermore, none of the current applications foster competition among the user, competition being understood as an effective tool for bring out one's best.

As can be seen, there is a need for a mobile fitness social media competition platform adapted to track and rank users against each other and professional and amateur athletes in a competitive manner providing an improvement in fitness-based online platforms through a practical application of an ordered combination of steps regarding the tracking, ranking and competing by way of fitness data associated with each user of the online social media platform.

The present invention is the only mobile fitness social media platform that tracks and ranks the strength of users around the world, and thereby enables the creation of competitive forums for urging users to give their peak performance. The platform is also adapted to track the strongest professional and amateur athletes and teams for hosting above-mentioned competitions. The globally attended competitions will attract fitness-related companies, such as supplement providers, providing a stream of advertisement revenue to further incentivizing users to compete for a prize money. Fitness-related companies can become partner-users, and their provided revenue may be in the form of cryptocurrency enabled by the systemic application. The systemic cryptocurrency can also be earned by posting fitness-related videos and fulfilling fitness-related achievements, further bolstering the self-sustaining nature of the underlying online platform. The cryptocurrency can be used on merchandise of the partner users. Additionally, the platform can track user's workouts, user's lifting accomplishments, user's food intake, count user's steps and the like so that users can compare their results with the fitness data associated with other users and professional and amateur famous provided through the online fitness-related platform.

### SUMMARY OF THE INVENTION

In one aspect of the present invention, a method of operating an online fitness-related social media platform over a network includes the following: providing a server of a social media platform system; providing, by the server, an application for execution on a user device connected to the network and associated with a user, the user device being remote from the server; providing a tracking functionality configured to track fitness data for each user device, providing a ranking functionality configured to rank one or more values associated with said fitness data; and operating the social media platform system in a competition of the method according to a set of rules applied in an ordered combination of steps, the set of rules comprising rules of: the user must request a challenge or accept the challenge another user to participate in the competition; each challenge associated with a wager of cryptocurrency only applicable on said network; setting a period of time for the tracking and ranking of the fitness data; and inputting one or more values related to the relevant fitness data for the period of time.

These and other features, aspects and advantages of the present invention will become better understood with reference to the following drawings, description and claims.

### BRIEF DESCRIPTION OF THE DRAWINGS

FIG. 1 is a block diagram of an exemplary embodiment of the present invention;

FIG. 2 is a first schematic view of an exemplary embodiment of the present invention;

FIG. 3 is a second schematic view of an exemplary embodiment of the present invention;

FIG. 4 is a third schematic view of an exemplary embodiment of the present invention; and

FIG. 5 is a fourth schematic view of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a method of operating an online fitness-related social media platform over a network includes the following: providing a server of a social media platform system; providing, by the server, an application for execution on a user device connected to the network and associated with a user, the user device being remote from the server; providing a tracking functionality configured to track fitness data for each user device, providing a ranking functionality configured to rank one or more values associated with said fitness data; and operating the social media platform system in a competition between users wherein their fitness data is tracked and ranked accordingly.

The ordered combination of various ad hoc and automated tasks in the presently disclosed platform necessarily achieve technological improvements through the specific processes described more in detail below. In addition, the unconventional and unique aspects of these specific automation processes represent a sharp contrast to merely providing a well-known or routine environment for performing a manual or mental task.

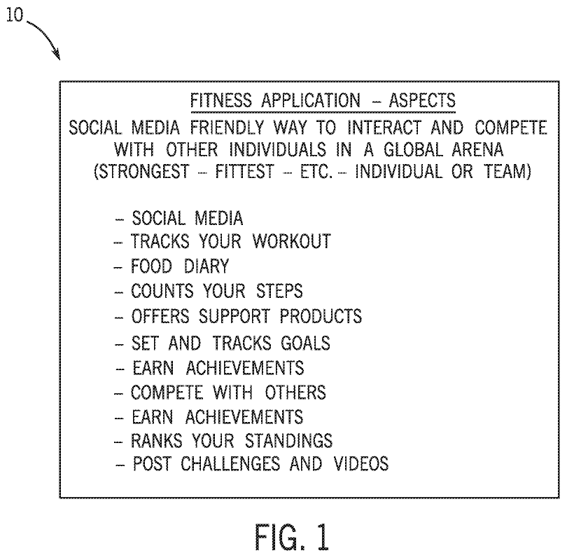

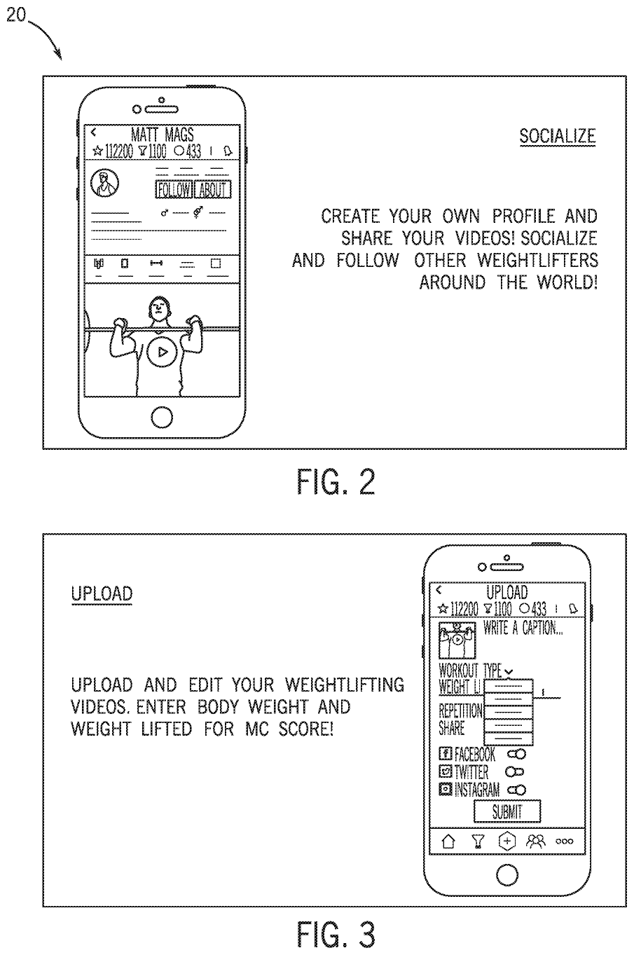

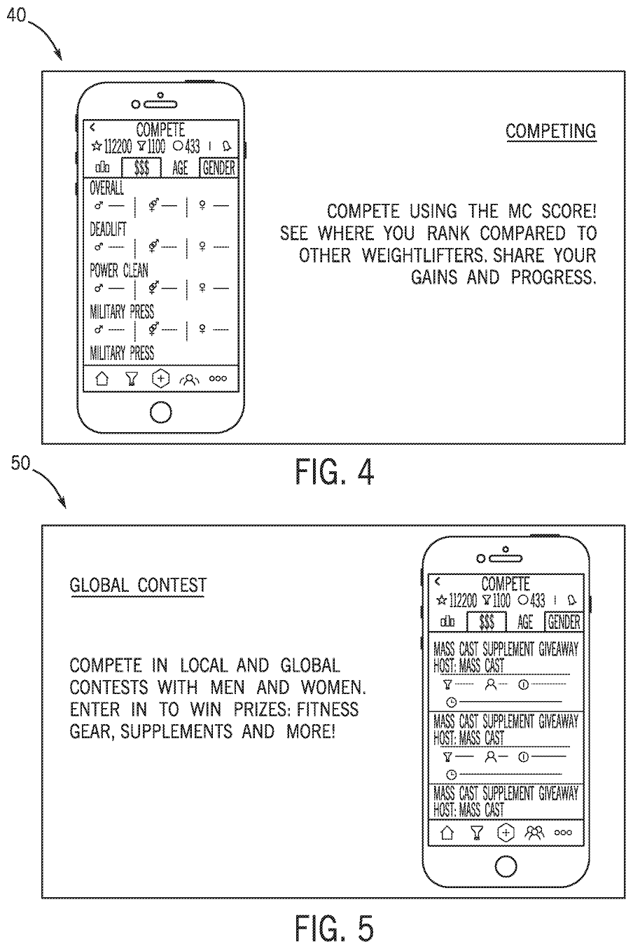

Referring to FIGS. 1 through 5, the present invention may include at least one computer with a user interface. The computer may include at least one processing unit coupled to a form of memory. The computer may include, but not limited to, a microprocessor, a server, a desktop, laptop, and smart device, such as, a tablet and smart phone. The computer includes a program product including a machine-readable program code for causing, when executed, the computer to perform steps. The program product may include software which may either be loaded onto the computer or accessed by the computer. The loaded software may include a systemic application 10 on the computer. The software may be accessed by the computer using a web browser. The computer may access the software via the web browser using the internet, extranet, intranet, host server, internet cloud and the like.

The systemic application 10 enables an online social media platform 20 that includes the creation of user profiles and associated profile output include fitness data. The online social media platform 20 also provides for partner-users that may include commercial or non-profit organizations. The systemic application 10 enables a tracking functionality for tracking the fitness data of other users, wherein fitness data may include but not be limited to workout numbers, dietary intake, set goals, athletic achievements, standings relative to other users and the like. For example, the present invention can track the amount of weights lifted, the number of repetitions of said weights lifted, the total amount of weight lifted during a given period of time, and the like. In another example, the systemic application 10 may track the strength of the users by using both the Wilk's Coefficient and Sinclair Coefficient to calculate the pound for pound/kilogram for kilogram of users and others associated with fitness data, for comparative analysis amongst the sets of fitness data associated with users and tracked athletes.

The online social media platform 20 enables the creation of a plurality of users into discrete teams in order to make team vs. team comparisons as well as related team-v-team competitions. For example, the present invention enables the formation of a team from a specific gym, say gym A, and so that gym A (a partner-user) can track and broadcast the results of, say, the total weight lifted (or any other portion of fitness data) by a first team from gym A against the total weight lifted of a second team from gym A. Thereby providing their member with more relevant fitness-related information, that is likely to be incentivizing because gym members would be competing against their own members, possibly in the same space. Likewise, the aggregated results of say, all the members from gym A can be tracked and compared against all the members from another gym, gym B. Similarly, all the resulting fitness data of the users associated with a first fitness instructor can be compared against the fitness data of users working with a second fitness instructor, to see which fitness instructor is getting the best results. Moreover, users can compare their individual fitness data statistics associated with the first fitness instructor relative to their statistics achieved by using the second instructor in an attempt to make an apples-to-apples comparison for which fitness instructor is best suited for them.

The online social media platform 20 is adapted to enable users to set goals, compare, rank, set and achieve fitness-date-related goals with other users, as well as post challenges and other audio-video output, and provide offerings of fitness-related products among and between users, including partner-users. For example, the present invention can rank users by the counted number of steps during an exercise routine, and recommend to each user the same fitness-related products used by another user with a higher counted number of steps that is closer or at the first user's set goal.

online social media platform 20 enables users to create their own profile and share their profile output, such as textual, audio and video output as well as fitness data that includes fitness-related achievements, with other users. More specifically, the profile output can be transmitted to other users in the form a challenges or goal-oriented offerings.

Once shared, a user's profile can be followed, tracked and ranked according to the above-mentioned tracking and ranking functionality 40. In other embodiments, by being a user, the entirety of non-confidential profile output may be shared among the users of the online social media platform 20. For example, weightlifters' competitive lifting results who are users can be tracked and ranked against users (around the world) of the online social media platform 20. Fitness data associated with professional and top amateur (college, high-school, etc.) athletes, who may not be users of the online social media platform 20, may be collected and filtered so that user of the online social media platform 20 can rank and compare themselves relative to these professional and amateur athletes. Furthermore, professionals and amateurs that are users of the online social media platform 20 can upload their fitness data results, as illustrated in FIG. 3, for exploitation through the tracking and ranking functionality 40. Additionally, the systemic application 10 can be used to filter the results of the tracking and ranking functionality 40; for instance, the strength of professional and amateur participants can be ranked by age groups, gender, and other demographic identifiers. This functionality is adapted to provoke users to tailor their comparisons to further incentivize them to achieve their fitness goals.

The tracking and ranking functionality 40 of the systemic application 10 further incentivizes users to share their gains and progress, and thus motivates the user to improve their results and thus their fitness. The systemic application 10, through the online social media platform 10, provides a competition functionality 50 for hosting global competitions, which are facilitated through the tracking and ranking functionality 40. The competitions enable users to compete in local and global contests so that users can compete for prizes and systemic cryptocurrency. The cryptocurrency may also be earned by using the online social mediate platform 20 (posting profile output, such as videos and earning achievements) and through winning competitions enabled through the competition functionality 50. The systemic cryptocurrency can be used on merchandise, including goods and service offered by partner-users, such as fitness supplements. For instance, supplement companies who are partner-users can advertise/giveaway their products to the contestants/users who buy into the competition-functionality 50 enabled tournaments through the associated systemic cryptocurrency. Also, users may gain cryptocurrency for posting on the online social media platform 10 and use the cryptocurrency towards competitions, merchandise, etc. The partner-user can also provide rewards and discount codes for their goods and services. The partner-user supplement companies and pay a fee for advertising their products but the systemic application 10 will remain free for users unless they choose to buy more cryptocurrency.

In short, the present invention, through the online social media platform 10 and associated functionalities, tracks and ranks the strength of users around the world. It has the ability to track the strongest teams, professional and amateur athletes, and hosts competitions where supplement companies can advertise/giveaway their products to the contestants/users who buy into these tournaments with the systemic cryptocurrency.

In additional embodiments, the systemic application 10 may implement a user voting functionality so users can be judged on their form by other users in additional to quantifiable fitness data such as pounds lifted or steps taken.

The computer-based data processing system and method described above is for purposes of example only, and may be implemented in any type of computer system or programming or processing environment, or in a computer program, alone or in conjunction with hardware. The present invention may also be implemented in software stored on a computer-readable medium and executed as a computer program on a general purpose or special purpose computer. For clarity, only those aspects of the system germane to the invention are described, and product details well known in the art are omitted. For the same reason, the computer hardware is not described in further detail. It should thus be understood that the invention is not limited to any specific computer language, program, or computer. It is further contemplated that the present invention may be run on a stand-alone computer system, or may be run from a server computer system that can be accessed by a plurality of client computer systems interconnected over an intranet network, or that is accessible to clients over the Internet. In addition, many embodiments of the present invention have application to a wide range of industries. To the extent the present application discloses a system, the method implemented by that system, as well as software stored on a computer-readable medium and executed as a computer program to perform the method on a general purpose or special purpose computer, are within the scope of the present invention. Further, to the extent the present application discloses a method, a system of apparatuses configured to implement the method are within the scope of the present invention.

It should be understood, of course, that the foregoing relates to exemplary embodiments of the invention and that modifications may be made without departing from the spirit and scope of the invention as set forth in the following claims.

What is claimed is:

1. A method of operating an online fitness-related social media platform over a network, comprising:
providing a server of a social media platform system;
providing, by the server, a software application for execution on a user device connected to the network and associated with a user, the user device being remote from the server;
providing a tracking functionality configured to track fitness data for each user device,
providing a ranking functionality configured to rank one or more values associated with said fitness data; and
operating the social media platform system in a competition of the method according to a set of rules applied in an ordered combination of steps,
the set of rules comprising rules of:
the user must request or accept a challenge with another user to participate in the competition;
each challenge associated with a wager of cryptocurrency only applicable on said network;
setting a period of time for the tracking and ranking of the fitness data; and
inputting one or more values related to the relevant fitness data for the period of time.

2. The method of claim 1, wherein the one or more values include pounds of weights lift.

3. The method of claim 1, wherein the one or more values include steps during an exercise routine.

4. The method of claim 1, further comprising a plurality of users, wherein two or more users are defined in one or more teams, and the one or more teams request or accept the challenge with another team.
